(19) United States
(12) Patent Application Publication
(10) Pub. No.: US 2002/0188228 A1
(43) Pub. Date: Dec. 12, 2002

(54) Chest cavity classifier apparatus and method for an artificial heart transplant

(76) Inventors: Douglas S. McNair, Gloucester, MA (US); Theophano Mitsa, Beverly, MA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 09/844,253

(22) Filed: Apr. 27, 2001

(51) Int. Cl. A61B 5/103; A61B 5/117

(52) U.S. Cl. 600/587

(57) ABSTRACT

An automated classifier receives input data indicative of a prospective surgical patient's mediastinal volume, body surface area and gender, and provides a classifier output signal indicative of quality of fit of a totally implantable artificial heart in the chest cavity of the patient. The present invention assists the surgical team in making their preoperative decision regarding whether or not the totally implantable artificial heart will fit in the chest cavity of the candidate patient. The classifier may also receive as input parameters: the distance between the patient's spine at the sternum at the level of the pulmonary bifurcation; the distance between the spine and the sternum in a caudal heart slice; the maximum left to right distance dimension of the heart; the distance from the rightmost end of the heart to the left chest wall; and the maximum left to right dimension of the chest cavity. Each of the input signals to the classifier is multiplied by an associated regression coefficient, and the resultant products are summed to provide a dependent variable whose value is indicative of quality of fit. To ensure that the classifier provides an accurate measure of quality of fit, the classifier coefficients are developed using a training process, and preferably verified in a testing process.

### BACKGROUND OF THE INVENTION

1. Field of Use

The present invention relates generally to cardiac assist devices and, in particular to an automated classifier for assessing preoperatively whether a cardiac assist device will fit within a patient's chest cavity.

2. Related Art

A totally implantable artificial heart offers the potential for an excellent quality of life for the recipient. Recent progress in modern technology, improvements in surgical techniques and increased understanding of circulatory physiology of cardiac assist device recipients indicate that a permanent mechanical replacement heart is now becoming a viable therapy for the treatment of patients having end-stage heart failure.

Realization of this potential requires minimization of the size and weight of the implantable elements including the blood pump assembly. Current design activities have focused on the most effective, anatomically compatible configuration of the blood pump, including the inflow and outflow ports. However, because the size, shape and topography of the anatomical structures of the chest cavity vary among patients, a particular blood pump will not fit into the chest cavity of all candidate patients.

Conventionally, surgical teams determined whether a candidate patient could receive a temporary cardiac assist device simply by performing sternotomy or other surgical procedure and comparing the physical dimension of the patient's chest cavity with the device. Recent advances in imaging technology have made available X-ray, MRI and/or CT images of the patient's anatomy. In an effort to avoid unnecessary surgery, such images of a patient's chest cavity are routinely reviewed before implanting a cardiac assist device. A similar approach can be used to make a determination as to anatomical fit of a total artificial heart device prior to surgery. Although viewing such images will likely result in an accurate decision for some candidate patients, its accuracy is quite limited. There are a host of patients which can be incorrectly accepted due to slight variations in anatomical structures that prevent the replacement heart device from fitting into the chest cavity. Although such variations can be accommodated during implantation of a cardiac assist device, there is less flexibility with a total replacement device.

In many instances, the patient's medical state is such that an MRI and/or CT image cannot be taken. In this case, often the only imaging technique available to the surgical team for assessing the patient's suitability for an artificial replacement heart is an X-ray image. The surgical team then makes various chest anatomical measurements from the X-ray images to determine whether or not the patient is a candidate for the replacement artificial heart. One technique for determining whether or not the patient is a candidate is disclosed in the paper entitled “Anatomic Fitting Studies of a Total Artificial Heart in Heart Transplant Receipt” by Fukamachi et al, ASAIO Journal 1996, 42:M337-M342. A problem with the fit technique disclosed in the paper by Fakamachi et al is that it looks strictly at an index value representative of volume. Specifically, this technique multiplies: (i) the craniocaudal distance from the dorsal region of the 8^{th} rib to the left diaphragm, (ii) the maximum left chest width and (iii) the maximum anteroposterior sternum-vertebrae dimension. The product of multiplying these three values together provides the index value, which is indicative of whether or not the patient is a good candidate for the replacement heart. A problem with this technique is that it is based upon a univariate decision process. That is, this paper discloses a technique that simply looks at only the volume of the patient's chest cavity using measurements obtained from X-ray images. Thus, if one of the dimensions is usually large, while a second dimension is unusually small, then the volume index value may indicate that the patient is a candidate for the transplant, when in fact the patient is not because of the a typical dimensions. In addition, the technique disclosed in the paper by Fukamachi et al fails to look at the anatomical dimensions of the patient's heart.

Therefore, there is a need for a reliable technique for determining whether or not a patient is a candidate for receiving the total implantable artificial heart.

### SUMMARY OF THE INVENTION

Briefly, according to an aspect of the present invention, an automated classifier receives input data indicative of a prospective surgical patient's mediastinal volume, body surface area and gender, and provides a classifier output signal indicative of quality of fit of a totally implantable artificial heart in the chest cavity of the patient.

The classifier may also receive as input parameters: the distance between the patient's spine at the sternum at the level of the pulmonary bifurcation; the distance between the spine and the sternum in a caudal heart slice; the maximum left to right distance dimension of the heart; the distance from the rightmost end of the heart to the left chest wall; and the maximum left to right dimension of the chest cavity. Each of the input signals to the classifier is multiplied by an associated regression coefficient, and the resultant products are summed to provide a dependent variable whose value is indicative of quality of fit.

To ensure that the classifier provides an accurate measure of quality of fit, the classifier coefficients are developed using a training process, and preferably verified in a testing process. The classifier is trained and tested using measurements from a plurality of patients for which the classification is known (e.g., fit, no fit or indeterminate).

In one embodiment the classifier is based upon on the partial-least squares model, which is an extension of multiple linear regression and its main idea is that the regression coefficients reflect the covariance between the predictor variables (i.e., the classifier input signals xᵢ) and dependent variable Y, which is the classifier output signal.

Advantageously, the present invention assists the surgical team in making their preoperative decision regarding whether or not the totally implantable artificial heart will fit in the chest cavity of the candidate patient.

These and other objects, features and advantages of the present invention will become apparent in light of the following detailed description of preferred embodiments thereof, as illustrated in the accompanying drawings.

### BRIEF DESCRIPTION OF THE DRAWINGS

FIG. 1 is a functional block diagram illustration of an automated classifier;

FIG. 2 is a perspective view of a biventricular artificial heart;

FIG. 3 is a flowchart illustration of a software routine associated with the automated classifier of FIG. 1;

FIG. 4 is a flow chart illustration of a classifier training process;

FIG. 5 is a flow chart illustration of a classifier testing process;

FIG. 6 is a block diagram illustration of the classifier processing;

FIG. 7 is a block diagram illustration of the classifier training process;

FIG. 8 is a block diagram illustration of the classifier testing process; and

FIG. 9 is a tabular illustration of regression coefficients computed during several classifier training processes.

### DETAILED DESCRIPTION OF THE INVENTION

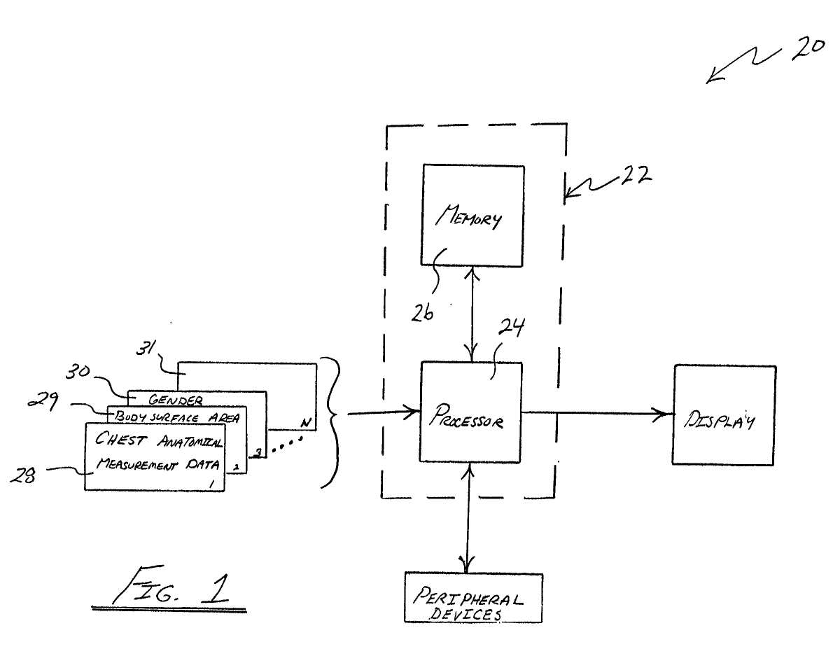

FIG. 1 is a functional block diagram illustration of an automated classifier 20 for assessing the quality of fit of a total implantable artificial heart in the chest cavity of a prospective surgical patient's chest cavity. The system 20 includes a workstation 22 comprising a processor 24 and memory 26 (e.g., disk). In one preferred embodiment the workstation may be a IBM compatible personal computer having at least one processor such as an Intel Pentium™ device. The workstation 22 receives a plurality of input signals 28-31 representative of the patient, including data indicative of the patient's chest cavity 28, the patient's body surface area (BSA) 29 and the patient's gender 30.

The classifier is preferably a regression classifier. As known, regression analysis is the description of the nature of the relationship between two or more variables, and is concerned with the problem of describing or estimating the value of the dependent variable on the basis of one or more independent variables.

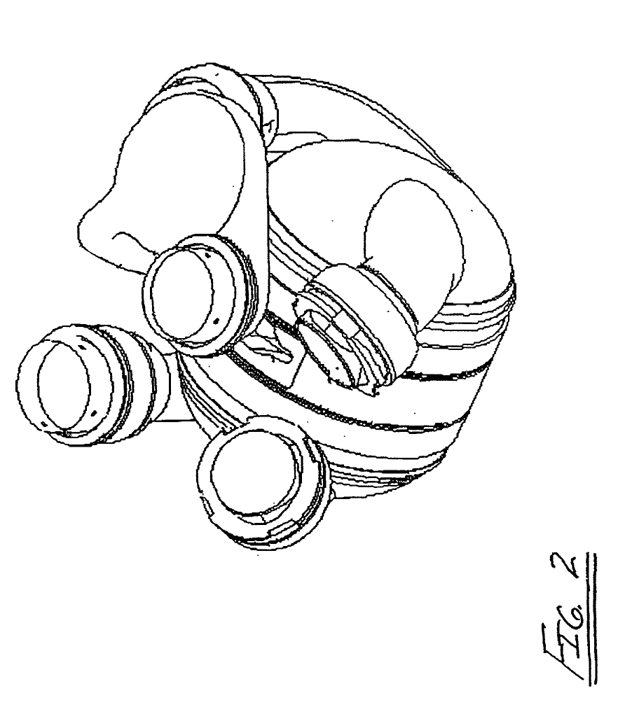

FIG. 2 is a perspective view of a totally implantable artificial heart 40. The automated classifier 20 (FIG. 1) operates on the data 28-31 (FIG. 1) representative of the patient to determine whether or not the implantable artificial heart 40 will fit within the chest cavity of the patient.

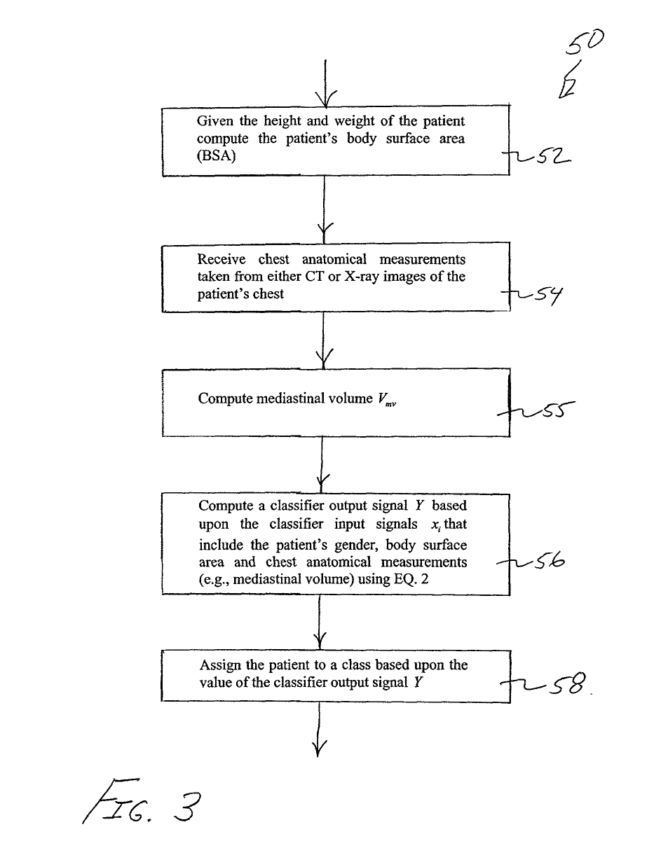

FIG. 3 is a flowchart illustration of a software routine 50 associated with the automated classifier of FIG. 1. The routine 50 is stored in the memory 26 (FIG. 1) and executed by the processor 24 (FIG. 1). The routine 50 includes a step 52 to compute a patient's BSA using the height and weight of the patient. Step 54 is then performed to receive various anatomical patient chest measurements from images (e.g., CT, MRI or X-ray) of the patient's chest cavity. It is contemplated that the large majority of the measurements will be taken from X-ray images due to the critical condition of the candidate patients who have a need for a total implantable artificial heart. In one embodiment, the anatomical patient chest measurements are determined by manually measuring the values from the X-ray images and inputting the measured values to a computer readable data file. Alternatively, it is contemplated that the anatomical measurement values may be automatically determined from the images (e.g., a digitized X-ray image), and made available in a computer readable data file.

The anatomical measurement values that are collected in the step 54 may include: (i) the distance between the patient's spine at the sternum at the level of the pulmonary bifurcation, (ii) the distance between the spine and the sternum in a caudal heart slice, (iii) the maximum left-to-right dimension of the chest cavity, (iv) the maximum left-to-right dimension of the heart, (v) the distance from the rightmost end of the heart to the left chest wall, and (vi) the distance from the pulmonary bifurcation to the caudal heart slice. Several of these measured values may be used in step 55 to compute the patient's mediastinal volume Vₘᵥ as follows:

Vₘᵥ=(ii)*(v)*(vi) EQ. 1

where operands (ii), (v) and (vi) are set forth in the preceding sentence. These measured and computed values are then used by the classifier 20 (FIG. 1) of the present invention to provide an indication of whether or not the totally implantable artificial heart will fit within a candidate patient's chest. Significantly, the patient's computed mediastinal volume Vₘᵥ is indicative of the size of the patient's heart.

Step 56 is performed next to compute a classifier output signal preferably based upon the input signals indicative of the patient's gender, BSA, and the patient's mediastinal volume Vₘᵥ. These inputs are preferred since they have been determined to be the most predictive of fit from: the group of inputs (i)-(vi) set forth in the preceding paragraph, gender, BSA, and mediastinal volume Vₘᵥ. However, of course you can use additional inputs in combination with the preferred input signals indicative of the patient's gender, BSA, and Vₘᵥ. How these three inputs were determined to be the most predictive of fit shall be discussed hereinbelow with respect to the classifier training process. Each of the input signals to the classifier is multiplied by an associated regression coefficient, and the resultant products are summed to provide a dependent variable Y (e.g., the classifier output signal) whose value is indicative of quality of fit. The dependent variable Y indicative of fit can be expressed as:

$$Y = {\sum }_{i = 0}^{N} {a}_{i} {x}_{i} \qquad \text{EQ. 2}$$

where:

aᵢ is the associated regression coefficient;

xᵢ is the associated input signal; and

N is the number of classifier input signals.

For example, in a preferred embodiment the inputs to the classifier are:

x₁=the patient's BSA;

x₂=the patient's mediastinal volume Vₘᵥ; and

x₃=patient gender, with female equal one and male equal two.

The patient's BSA may be computed using the following equation:

$$BSA = \frac{{e}^{( 0.425 * ( LN ( weight\_in \_kg ) + 0.725 * LN ( height\_in \_cm ) + LN ( 71.84 ) )}}{{10}^{4}} \qquad \text{EQ. 3}$$

The mediastinal volume Vₘᵥ is computed using the expression set forth in EQ. 1. The input signals xᵢ are then multiplied by their associated regression coefficients and the resultant products are summed to compute the classifier output signal value Y, as set forth in EQ. 2.

The classifier output signal Y is a real number that may be processed (e.g., rounded) to provide an integer value that is indicative of the quality of fit (i.e., the class of fit). For example, the classes may include: (i) fit, (ii) no-fit and (iii) indeterminate, wherein the value of the classifier output signal Y determines the class. In one embodiment, the value zero may be associated with no-fit, the value one may be associated with indeterminate, and the value two may be associated with fit.

We shall now discuss how the regression coefficients aᵢ are determined in a classifier training process.

Classifier Training

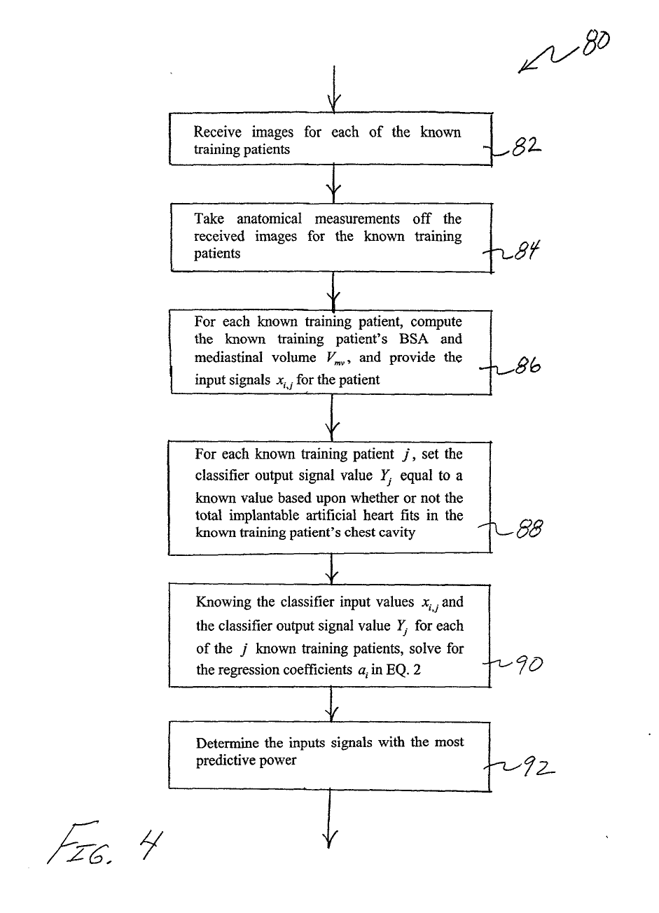

Before the classifier can be used to determine whether or not the artificial replacement heart fits within the chest cavity, the regression coefficients aᵢ (see EQ. 2) must be determined. This process involves solving for the classifier regression coefficients aᵢ of EQ. 2, knowing the value of the output signal Y and the input signals xᵢ for a plurality of patients. Referring to FIG. 4, training process 80 performs a first step 82 of receiving images (e.g., CT, MRI, or X-ray, etc) from patients (“known training patients”) for whom it is known whether or not the artificial heart will fit within their chest cavity. Step 84 is performed next to obtain anatomical measurements from these images. These measurements include: (i) the distance between the patient's spine at the sternum at the level of the pulmonary bifurcation, (ii) the distance between the spine and the sternum in the caudal heart slice, (iii) the maximum left-to-right dimension of the chest cavity, (iv) the maximum left-to-right dimension of the heart, (v) the distance from the rightmost end of the heart to the left chest wall, and (vi) the distance from the pulmonary bifurcation to the caudal heart slice. In step 86, for each of the known training patient's, the patient's mediastinal volume Vₘᵥ is computed using the expression set forth in EQ. 1. This step also computes the patient's BSA. The patient's mediastinal volume and BSA are input signals to the classifier.

For each of the known training patients whose images are used in steps 82-86, it is known whether or not the total replacement heart will fit into their chest. One technique for determining whether or not the total replacement heart will fit is obviously to actually surgically implant it, and build a database of known training patients based upon the results. Of course, this is not possible for the assessment of initial candidate implant patients (e.g., during clinical trials) since there are no patients who have yet actually undergone the total replacement heart implant. Therefore, at least initially, a database of known training patients will be developed by reviewing the chest cavity images of a number of cardiac patients, and from the images manually make a decision whether or not the total replacement heart will fit. This process may involve several trained individuals reviewing the images and making the decision of whether or not the implantable replacement heart would fit. In addition, each of the trained individuals preferably performs multiple reviews (e.g., over a period of days) of a particular patient's images to decrease the probability that the trained individual is making an incorrect decision regarding fit. For example, for each patient five experts may review the images to determine whether or not the total replacement heart will fit, and then the votes from the five experts are tallied to determine what the majority decision is. In addition to manually viewing the training images to determine whether the artificial replacement heart fits, the technique disclosed in U.S. patent application Ser. No. 09/726,635, entitled “Computer-Aided Apparatus and Method for Preoperatively Assessing Anatomical Fit of a Cardiac Assist Device Within a Chest Cavity”, filed Nov. 30, 2000 may also be used by the experts to provide an indication of whether the replacement heart will fit within the chest cavity of the patient.

Referring again to step 88, for each of the known training patients that the heart fits into, the dependent value Y is set to a first known value (e.g., equal to two). For each of the known training patients that the heart does not fit into, the dependent value Y is set to a second known value (e.g., equal to zero). For each of the known training patients that it is indeterminate whether or not the artificial heart will fit, the dependent value Y is set to a third known value (e.g., equal to one). Thus, at this point in the training process the value of the dependent value Y and the associated input signals xᵢ are known for each of the known training patients. Therefore, an expression in the form of EQ. 2 can be provided for each of the known training patients. That is, EQ. 2 can be rewritten for each of the j number of known training patients as:

$${Y}_{j} = {\sum }_{i = 0}^{N} {a}_{i , j} {x}_{i , j} \qquad \text{EQ. 4}$$

Since the values Yⱼ and x_{i,j} are known for each of the training patients, the training process processes the j number of expressions set forth in EQ. 4, or a sub-set of the j number of expressions, to determine the regression coefficients aᵢ to be used in the classifier expression set forth in EQ. 2.

The training process 80 may also include a correlation step 89, which determines the independent variables that have the highest predictive ability with respect to determining whether or not the artificial replacement heart will fit. For example, there may be eight available input signals (i.e., independent variables) for each patient that are used to compute the regression coefficients, but only several of these inputs may have a relatively high predictive ability with respect to determining whether or not the artificial replacement heart will fit. Significantly, it was determined that the three inputs most predictive of fit were patient gender, BSA and mediastinal volume Vₘᵥ. Therefore, in a preferred embodiment only the inputs determined to be the most predictive by the correlation step are used by the training process to compute the regression coefficients (e.g., N equals two in EQ. 4).

Since the values Yⱼ and x_{i,j} are known for each of the training patients, step 90 processes the sub-set of the j number of expressions associated with the most predictive inputs (e.g., the patient gender, BSA and mediastinal volume Vₘᵥ) to determine the regression coefficients aᵢ to be used in the classifier expression set forth in EQ. 2. That is, using the most predictive input signals xᵢ and whether or not the implantable replacement heart fits for each of the j number of known training patients (i.e., Yᵢ), step 90 solves for the regression coefficients αᵢ, (FIG. 2).

The regression coefficients aᵢ are computed using multiple regression. Multiple regression is a statistical analysis technique that is used to describe the relationship between several independent variables (predictors) and one dependent variable. Multiple regression identifies the linear equation that best predicts the value of the dependent variable. A popular implementation of multiple regression is stepwise regression. As known, stepwise regression involves: (i) definition of a convergence criteria, (ii) initial model identification, (iii) iterative model alteration based on the convergence criteria, and (iv) search termination when stepping is not possible given the convergence criteria of when a specified number of steps has been reached. In the present invention, the independent variables include both continuous variables (i.e., the anatomical measurements and the mediastinal volume Vₘᵥ) and categorical variables (e.g., gender). Therefore, a regression technique that operates on both continuous variables and categorical variables should be used. For example, in one embodiment a suitable stepwise regression is based on analysis of covariance. Alternatively, rather than analysis of covariance, another technique is homogeneity of slopes method. A difference between these techniques is that the homogeneity of slopes method tests for interactions between the independent variables, while the analysis of covariance does not. Both techniques have been used in the present invention with comparable results. Testing for interaction between variables provides in general a better regression model, since it allows for testing of interaction between independent variables such as gender, BSA and the anatomical measurements.

Referring still to step 90, the computation of the regression coefficients ak is preferably performed using a commercial-off-the-shelf software package, such as the STATISTICA™ application program available from StatSoft, Inc. (www.statsoftinc.com). CLASSIFIER TESTING

Once the classifier training is completed, the classifier may be tested against known images to verify the classifier operation. In this step, a plurality of images from patients (i.e., “known testing patients”) are used. Similar to the database of known training patients, at least initially, a database of known testing patients will be developed by reviewing the chest cavity images of a number of cardiac patients, and from the images make a decision whether or not the total replacement heart will fit from the images. This process preferably involves several trained individuals reviewing the images and making the decision. In addition, each of the trained individuals preferably performs multiple reviews (e.g., over a period of days) of a particular patient's images. In addition, the technique disclosed in U.S. patent application Ser. No. 09/726,635, entitled “Computer-Aided Apparatus and Method for Preoperatively Assessing Anatomical Fit of a Cardiac Assist Device Within a Chest Cavity”, filed Nov. 30, 2000 may also be used to provide an indication of whether the replacement heart will fit within the chest cavity of the patient. Once data from patients who have undergone the surgical implant procedure becomes available, the actual implant data patient may be added to the known testing patient database. For each of the testing patients it is known whether or not the total replacement artificial heart fits. The images used in this testing step are preferably different than the images used in the classifier training step. However, it is contemplated that some of the images used in the training step may be used in this testing step since the number of available images may be limited.

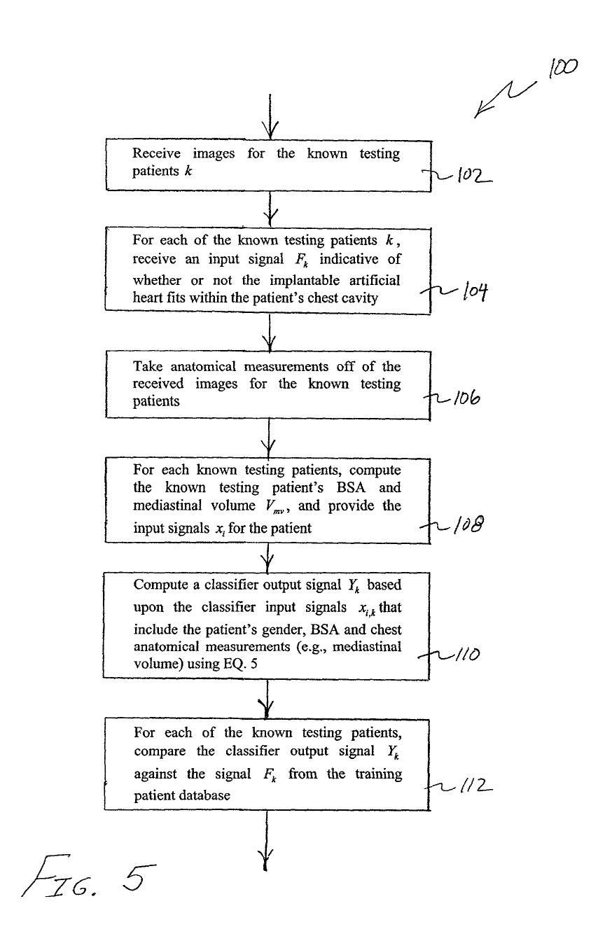

FIG. 5 is a flow chart illustration of a classifier testing process 100. The testing process includes a first step 102 to receive various chest images of the k number of testing patients. As set forth above, for each of the k number of testing patients it is known whether or not the artificial heart fits. Thus, in step 104 for each of the k testing patient's an input signal Fₖ is received from the testing patient database indicative of whether or not the artificial heart fits within the patient's chest. Step 106 is performed next to take anatomical measurements off of the received images for the known testing patients. As set forth above, these measurements include: (i) the distance between the patient's spine at the sternum at the level of the pulmonary bifurcation, (ii) the distance between the spine and the sternum in a caudal heart slice, (iii) the maximum left-to-right dimension of the chest cavity, (iv) the maximum left-to-right dimension of the heart, (v) the distance from the rightmost end of the heart to the left chest wall, and (vi) the distance from the pulmonary bifurcation to the caudal heart slice.

Step 108 is then performed to compute the patient's BSA and mediastinal volume Vₘᵥ, and provide the input signals xᵢ for the patient. Using the expression set forth in EQ. 5, step 110 computes a classifier output signal Yₖ for each of the k number of testing patients using at least the patient's gender, BSA, and anatomical information (e.g., the patient's mediastinal volume Vₘᵥ).

$${Y}_{k} = {\sum }_{l = 0}^{M} {a}_{l} {x}_{l , k} \qquad \text{EQ. 5}$$

where:

aᵢ is the associated regression coefficient;

xᵢ is the associated input signal; and

M is the number of classifier input signals.

The regression coefficients aᵢ used in EQ. 5 are those computed in step 90 in FIG. 4. Step 112 is then performed to compare each computed classifier output signal Yₖ against the input signal Fₖ from the testing patient database indicative of whether or not the artificial heart fit within the patient's chest. That is, for each of the k training patients, step 112 compares the classifier output signal Yₖ to the input signal Fₖ. If the classifier output signal Yₖ indicates that the artificial heart will fit, while the input signal Fₖ indicates that the heart does not fit, then a regression coefficient error condition exists. This comparison is made for each of the k number of training patients.

If the comparisons performed in step 112 indicate poor correlation between the classifier output signals Yₖ against the input signals Fₖ then regression coefficients computed in step 90 (FIG. 4) need to be adjusted, since the testing process indicates that the classifier outputs using the regression coefficients computed in step 90 (FIG. 4) are not matching the known testing patient data. Similarly, if the comparisons performed in step 112 indicate good correlation between the classifier output signals Yₖ and the input signals Fₖ, then the regression coefficients computed in step 90 (FIG. 4) are valid.

One of ordinary skill will recognize that the testing step 100 (FIG. 5) is certainly not required, but rather it is a step in one embodiment of the classifier development process that verifies the classifier configuration utilizing the computed regression coefficients from step 90 (FIG. 4) provides valid results.

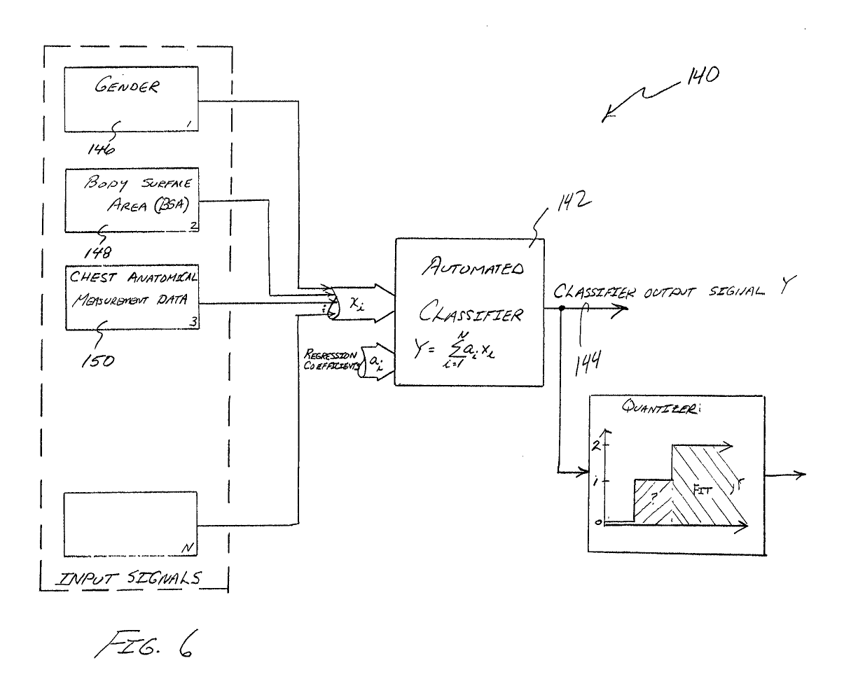

FIG. 6 is a block diagram illustration of a classifier processing system 140. Automated classifier 142 receives a plurality of input signals, and computes and provides a classifier output signal Y on a line 144. The output signal may be provided to a display device or other conventional output peripheral device. The input signals include a plurality of patient specific input signals xᵢ and a plurality of regression coefficients aᵢ. The plurality of patient specific input signals xᵢ include patient gender 146, patient BSA 148, patient mediastinal chest volume Vₘᵥ 150, etc. The regression coefficients aᵢ are those computed step 90 (FIG. 2). As set forth above, the automated classifier 142 is preferably an executable routine that is stored in system memory and executed by a processor (e.g., a CPU). In one embodiment, the classifier may be implemented in the workstation 22 (FIG. 1), such as an IBM™ compatible personal computer having at least one processor. The classifier system 140 may also include a quantizer that receives the output signal Yon the line 144, which is preferably a real number, and rounds the value to provide an integer output value indicative of fit.

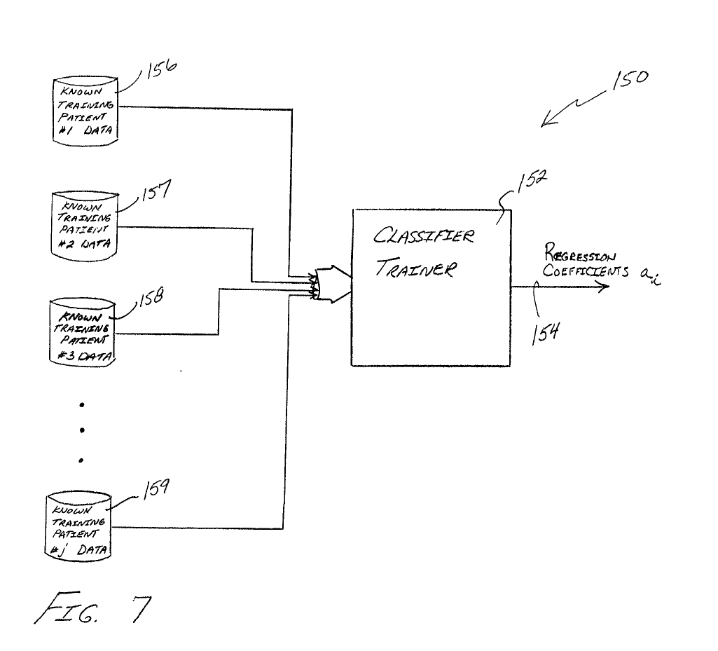

FIG. 7 is a block diagram illustration of a classifier training mechanism 150. The training mechanism 150 includes a classifier trainer 152 that receives data from a plurality of training patients, and computes and provides classifier regression coefficients aᵢ that are provided on a line 154 and stored. The training mechanism 150 receives known training patient data 156-159. The classifier trainer 152 executes a classifier training routine (e.g., see FIG. 4), that uses the data from the known training patients to determine the regression coefficients that classify patients into predetermined fit categories (e.g., fit, no-fit, indeterminate).

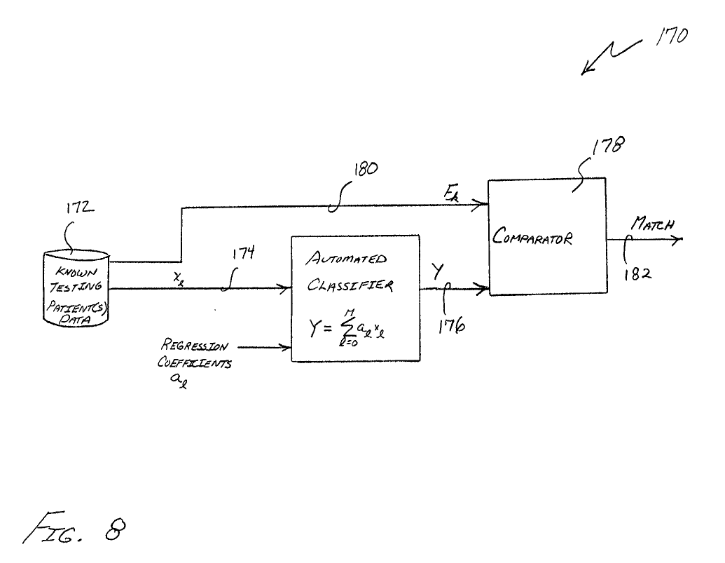

FIG. 8 is a block diagram illustration of the classifier testing system 170. The system receives known testing patient data 172 that includes input signals xᵢ on a line 174 indicative of the known testing patient's mediastinal volume Vₘᵥ, BSA and gender, and provides a classifier output signal Y on a line 176 indicative of quality of fit of a totally implantable artificial heart in the chest cavity of the patient. The classifier output signal Yon the line 176 is input to a comparator 178, which also receives the input signal Fₖ on a line 180 that is indicative of whether or not the artificial heart (FIG. 2) fits. The comparator 178 compares the signals on the lines 176 and 180 to verify that they indicate the same classification. The comparator 178 provides a Boolean output signal on a line 182 indicative of whether or not the signals 176 and 180 indicate the same classification. This routine is preferably performed for a number of known testing patients to properly test the classifier coefficients. We shall now present an example of the training process calculation.

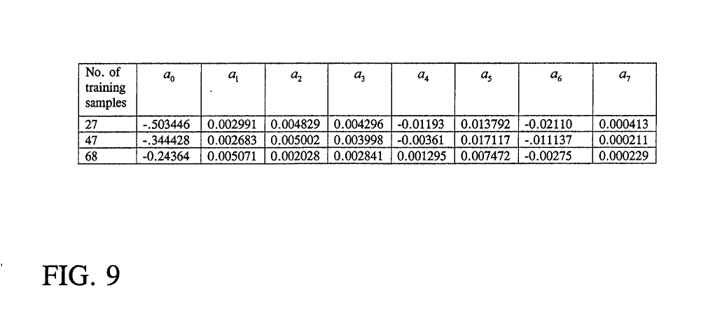

FIG. 9 is a tabular illustration of regression coefficients computed during several classifier training processes. In a first row of the table, data from twenty-seven (27) training patients were used to compute the regression coefficients. The data for each of the training patients used to compute the regression coefficients included:

x₀=gender;

x₁=(BSA×100);

x₂=the distance in millimeters between the patient's spine at the sternum at the level of the pulmonary bifurcation;

x₃ the distance in millimeters between the spine and the sternum in a caudal heart slice;

x₄=the maximum left-to-right dimension in millimeters of the heart;

x₅=the distance in millimeters from the rightmost end of the heart to the left chest wall;

x₆=the maximum left-to-right dimension in millimeters of the chest cavity; and

x₇=the computed mediastinal volume Vₘᵥ in cubic millimeters.

Note, the values for x₃ x₅ and x₆ are preferably determined from the same image slice. Using the inputs indicative of patient gender, BSA and mediastinal volume Vₘᵥ (i.e., the inputs deemed to be the most predictive) and the information from the training patient database regarding whether or not the replacement heart fits, step 90 in FIG. 4 was performed to determine the regression coefficients. The results are shown in the first row of the table in FIG. 9. The training process was performed again using data from forty-seven (47) training patients and the resultant regression coefficients are shown in the second row of FIG. 9. The training process was performed yet again using data from sixty-eight training patients and the resultant regression coefficients are shown in the third row of FIG. 9.

One of ordinary skill in the art will recognize that as the training patient database changes, the signals determined in the training process to be the most predictive may change. That is, it is contemplated that signals other than patient gender, BSA and mediastinal volume Vₘᵥ may be used to compute the regression coefficients, and as inputs to the regression classifier. Of course, the larger the training patient database, the more accurate the predictive power computations becomes.

It is contemplated that the surgical team may use the present invention in combination with another technique developed by the assignee of the present invention and disclosed in U.S. patent Ser. No. 09/726,635, entitled “Computer-Aided Apparatus and Method for Preoperatively Assessing Anatomical Fit of a Cardiac Assist Device Within a Chest Cavity”. This application is incorporated herein by reference. If there are CT or MRI images available of patient's chest cavity, then the surgical team may use the computer-aided apparatus and method disclosed in this co-pending application to assess whether or not the replacement heart will fit in the chest cavity. If there are only x-ray images available of patient's chest cavity, then the surgical may use only the present invention to assess whether or not the total implantable replacement device will fit within the chest cavity. Certainly, the surgical team may elect to use both the technique of the present invention and the technique disclosed in the above identified co-pending application to assist in the fit assessment.

One of ordinary skill will also recognize that the regression classifier discussed herein does not have any cross terms (i.e., it is first order). For example, none of the input signals are multiplied together in the classifier. It is contemplated that these additional cross terms may be added to the classifier expression (e.g., EQ. 2) in order to account for interactions between the predictors. For example, assuming a classifier with three independent variables xⱼ (i.e., where j=0−2) the regression equation that takes into account interactions has the form of:

Y=a₀+a₁x₁+a₂x₂+a₃x₃+a₄x₁x₂+a₅x₁x₃+a₆x₂x₃+a₇x₁x₂x₃ EQ. 6

The homogeneity of slopes method yields an equation similar to EQ. 6, while analysis of covariance yields an equation that does not include cross terms.

Advantageously, the present invention assists the surgical team in making their preoperative decision regarding whether or not the biventricular artificial heart will fit in the chest cavity of the candidate patient.

Although the present invention has been shown and described with respect to several preferred embodiments thereof, various changes, omissions and additions to the form and detail thereof, may be made therein, without departing from the spirit and scope of the invention.

What is claimed is:

1. A method of assessing if an implantable replacement artificial heart fits in a candidate patient's chest, comprising:
receiving signal values indicative of the surgical patient's mediastinal volume, body surface area and gender; and
processing, in a automated classifier, said signal values indicative of the surgical patient's mediastinal volume, body surface area and gender, to provide a classifier output signal indicative of whether or not the implantable replacement artificial heart will operatively fit in the chest cavity of the patient.

2. The method of claim 1, wherein said classifier comprises a regression classifier that includes a regression coefficient for each of said input signals, wherein said step of processing comprises multiplying each said received signal values by its associated said regression coefficient and summing the resultant products to provide said classifier output signal.

3. The method of claim 2, comprising receiving X-ray images and performing measurements to determine the surgical patient's mediastinal volume and body surface area.

4. The method of claim 1, wherein said step of receiving signal values indicative of the surgical patient's mediastinal volume comprises determining the mediastinal volume from patient X-ray images.

5. The method of claim 1, further comprising the step of quantizing said classifier output signal to provide a quantized classifier output signal value indicative thereof.

6. The method of claim 1, wherein in addition to said signals indicative of the surgical patient's mediastinal volume, body surface area and gender, said step of processing also processes at least one additional signal selected from the group of signal values comprising (i) the distance between the patient's spine at the sternum at the level of the pulmonary bifurcation, (ii) the maximum left-to-right dimension of the heart, (iii) the distance from the rightmost end of the heart to the left chest wall, and (iv) the maximum left-to-right dimension of the chest cavity, to compute said classifier output signal.

7. A system for assessing if an implantable replacement artificial heart fits in a candidate patient's chest, said system comprising:
means for receiving signal values indicative of the surgical patient's mediastinal volume, body surface area and gender; and
a processing device that includes a classifier responsive to said signal values indicative of the surgical patient's mediastinal volume, body surface area and gender, wherein said classifier computes a classifier output signal indicative of whether or not the implantable replacement artificial heart fits in the chest cavity of the patient.

8. The system of claim 7, wherein said classifier comprises
means for multiplying each of said input signals with an associated coefficient value, and for summing each of the resultant products to provide a summed value indicative of said classifier output signal.

9. The system of claim 8, wherein said means for receiving receives input signals selected from the group of signal values comprising (i) the distance between the patient's spine at the sternum at the level of the pulmonary bifurcation, (ii) the maximum left-to-right dimension of the heart, (iii) the distance from the rightmost end of the heart to the left chest wall, and (iv) the maximum left-to-right dimension of the chest cavity.

10. The system of claim 8, wherein said processing device includes a microprocessor that executes executable instructions to calculate said classifier output signal.

11. The system of claim 10, wherein said classifier includes a quantizer that receives and quantizes said classifier output signal value to provide a quantized classifier output signal value.

12. The system of claim 9, wherein said processing device also receives at least one additional signal selected from the group of signal values comprising (i) the distance between the patient's spine at the sternum at the level of the pulmonary bifurcation, (ii) the maximum left-to-right dimension of the heart, (iii) the distance from the rightmost end of the heart to the left chest wall, and (iv) the maximum left-to-right dimension of the chest cavity, and computes said classifier output signal using said at least one selected signal and said signal values indicative of the surgical patient's mediastinal volume, body surface area and gender.

13. A system for assessing if an implantable replacement artificial heart fits in a candidate patient's chest, said system comprising:
means for receiving signal values indicative of the surgical patient's mediastinal volume, body surface area and gender;
a database of regression coefficient values; and
means, responsive to said signal values indicative of the surgical patient's mediastinal volume, body surface area and gender, for receiving regression coefficient data from said database of regression coefficient values, and for computing a classifier output signal indicative of whether or not the implantable replacement heart fits in the chest cavity of the patient by computing the product of each of said signal values with their uniquely associated said regression coefficient value and for summing the resultant products to provide said classifier output signal.

14. A method of determining regression coefficients for use in a automated classifier that provides an indication of whether an implantable replacement artificial heart fits in a candidate patient's chest, said method comprising:
providing a database of known training patient data that includes, for each of a plurality of cardiac patients, data indicative of the patient's (i) gender, (ii) body surface area, (iii) mediastinal volume Vₘᵥ, and (iv) whether the implantable replacement heart fits within the patient's chest; and
computing said regression coefficients using said known training patient data, wherein each of said regression coefficients is uniquely associated with a selected one of the independent variable inputs indicative of gender, body surface area, and mediastinal volume Vₘᵥ.

15. The method of claim 14, wherein said step of computing includes computing the regression coefficients with a multiple regression computation.

16. The method of claim 14, wherein said step of computing includes computing the regression coefficients with a stepwise multiple regression computation.

17. The method claim 14, wherein said classifier is of the form
$$Y = {\sum }_{i} {a}_{i} {x}_{i} ,$$
where Y is indicative of said classifier output signal, aᵢ is indicative of said regression coefficients and xᵢ is indicative of the independent input variables.

18. The method of claim 14, further comprising
testing said coefficient values by comparing, using known testing patient data that includes a plurality of independent input variables and a dependent variable, a classifier output signal value that is computed by multiplying each of said independent input variables with a uniquely associated one of said coefficient values and summing the resultant products to provide a classifier test output signal that is compared to said dependent variable to determine if said classifier test output signal matches said dependent variable.
